(19) United States
(12) Patent Application Publication
(10) Pub. No.: US 2024/0108278 A1
(43) Pub. Date: Apr. 4, 2024

(54) COOPERATIVE LONGITUDINAL SKIN CARE MONITORING

(71) Applicant: Johnson & Johnson Consumer Inc., Skillman, NJ (US)

(72) Inventors: Logan McGill, Philadelphia, PA (US); Natacha Leduc, Toronto (CA)

(21) Appl. No.: 18/373,137

(22) Filed: Sep. 26, 2023

Related U.S. Application Data
(60) Provisional application No. 63/411,267, filed on Sep. 29, 2022.

(51) Int. Cl. A61B 5/00; G16H 10/20; G16H 10/60; G16H 50/20; G16H 80/00

(52) U.S. Cl. CPC A61B 5/441; A61B 5/0077; A61B 5/7465; G16H 10/20; G16H 10/60; G16H 50/20; G16H 80/00

(57) ABSTRACT

A tool that enables professional-level monitoring at home can provide patients with better access to expert advice regarding skin care. The tool may enable providing information about skin health to a patient's skin care specialist such that the most relevant information is emphasized and presented in a manner expected. The device may include a processor configured to receive a first skin image at a first time and a second skin image at a second time. The first and second time may be separated by a duration associated with a skin event. A plurality of skin characteristics may be determined from the first skin image and the second skin image. The processor may be configured to generate an analysis output and to transmit the analysis output to one or more receivers. The analysis output may include a synoptic representation of one or more of the plurality of skin characteristics.

### CROSS-REFERENCE TO RELATED APPLICATIONS

This application claims the benefit of U.S. Provisional Application No. 63/411,267, filed Sep. 29, 2022, the contents of which is incorporated by reference herein in its entirety.

### BACKGROUND

Proper skin care can be beneficial to reducing signs of aging, reducing acne, and improving overall health. While individuals may have access to skin care information and tools, they generally lack the expertise of a dermatologist. Dermatologists and other skin care specialists can provide expertise and tools for improving skin health. However, access to dermatologists is limited (e.g., based on appointment availability, prohibitive cost, etc.). Moreover, because a dermatologist cannot continuously monitor a patient, the dermatologist's ability to evaluate the health of a patient's skin can be limited to the information available at the time of the appointment.

### SUMMARY

A device and/or tool that enables skin health monitoring at home or in an office setting can provide patients with better access to expert advice regarding skin care. In some embodiments, the monitoring can occur once, occasionally, or continuously. In particular, the device and/or tool may enable providing information about skin health to a patient's skin care specialist such that the most relevant information is emphasized and presented in a manner expected.

The device may include a processor. The processor may be configured to receive a first skin image at a first time and a second skin image at a second time. The first skin image and the second skin image may be associated with a user. And the first and second time may be separated by a duration associated with a skin event.

The processor may be configured to determine a plurality of skin characteristics from the first skin image and the second skin image. For example, a skin characteristic of the plurality of skin characteristics may represent at least a skin element and a score associated with the skin element.

The processor may be configured to generate an analysis output. The analysis output may be based on an analysis configuration and on the plurality of skin characteristics. The analysis output may include a synoptic representation of one or more of the plurality of skin characteristics.

The processor may be configured to transmit the analysis output to one or more receivers. For example, the one or more receivers may include a skin care specialist. And the synoptic representation may present a historical summary of the one or more of the plurality of skin characteristics in a form suitable to the skin care specialist. For example, the analysis output may include a historical summary of the plurality of skin characteristics, representing a difference between a first skin characteristic associated with the first time and a second skin characteristic associated with the second time.

### BRIEF DESCRIPTION OF THE DRAWINGS

FIGS. 1-4 are user interface (UI) examples illustrating techniques for monitoring skin health or care over time.

FIG. 5 is an example timeline illustrating the collection of longitudinal information about a plurality of skin characteristics, generation of an analysis output, and transmission of the analysis output.

FIG. 6 is a flow diagram illustrating an example computer-implemented method.

FIG. 7 is a block diagram illustrating an generation of an analysis output based on longitudinal information about one or more skin characteristics.

FIGS. 8A & B illustrate an example analysis output.

FIG. 9 is a block diagram illustrating an example computing device.

### DETAILED DESCRIPTION

Proper skin care can be beneficial to reducing signs of aging, reducing acne, and improving overall health. While individuals may have access to skin care information and tools, they generally lack the expertise of a dermatologist. Dermatologists and other skin care specialists can provide expertise and tools for improving skin health. However, access to dermatologists is limited (e.g., based on appointment availability, prohibitive cost, etc.).

Moreover, because a dermatologist cannot continuously monitor a patient, the dermatologist's ability to evaluate the health of a patient's skin can be limited to the information available at the time of the appointment.

Accordingly, patients may benefit from a device or tool that enables skin health monitoring at home. Such a device may provide patients with better access to expert advice regarding skin care. Such a device may be capable of providing longitudinal information (e.g., information collected over a period of time) to a skin care specialist (e.g., a dermatologist). The longitudinal information may enable the skin care specialist to provide more insightful skin care recommendations to patients.

Because traditional dermatologist imaging is limited to premises-based equipment, appropriate interim imaging is unavailable. Conventional at-home imaging does not provide adequate analysis and information to be useful to a dermatologist. For example, a dermatologist who receives a large quantity of interim images, in typical color, digital form, from a user, does not have the technical means to organize, map, compare, and/or analyze such images. The approach disclosed herein addresses this technical issue by analyzing interim images and compressing the results to a synoptic representation of one or more of the plurality of skin characteristics associated with an area of concern of the user, e.g., for transmission to the dermatologist.

The device may include a processor configured to receive a first skin image and second skin image and determine a plurality of skin characteristics based on the first and second skin images. In some examples, one or more of the plurality of skin characteristics may be associated with an area of concern of the user. In some examples, the device may generate an analysis output including a synoptic representation that presents a historical summary of the one or more of the plurality of skin characteristics. The synoptic representation may be in a form that is suitable to a skin care specialist. For example, the synoptic representation may be in a form that will allow the skin care specialist to quickly determine an appropriate treatment for the user.

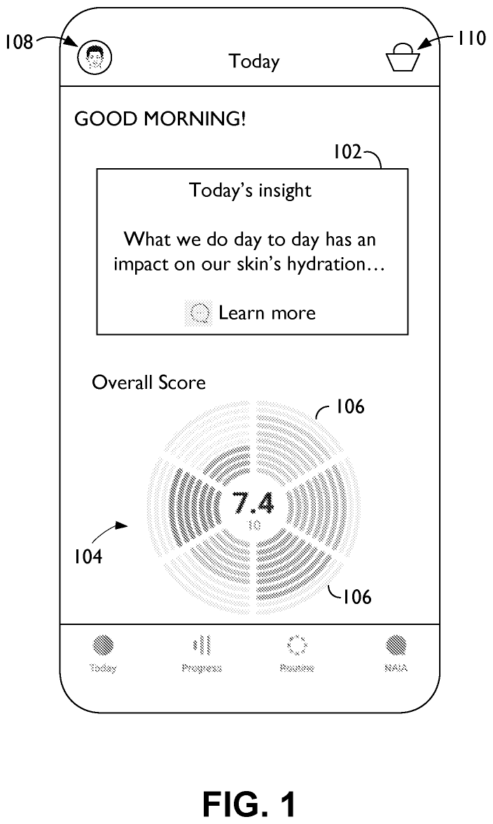

FIG. 1 is an example user interface (UI) illustrating a home screen of a mobile application for monitoring skin health or care over time. A person of ordinary skill in the art will appreciate that the UIs described herein may be implemented in ways other than a mobile application. For example, the UIs described herein may be implemented as a desktop (e.g., computer) application, a web-based application, or the like. As used herein, the term “application” may be used broadly to describe all of the possible ways that the UI may be accessed by a user.

Using a digital camera, a user may first acquire a skin image of the user's face. In some examples, acquiring a skin image may include acquiring a series of skin images. For example, acquiring a skin image may include acquiring a front-view skin image, a right-side skin image, and a left-side skin image of the user's face. In some examples, the skin image(s) may be acquired via picture capture. In some examples, the skin image(s) may be acquired via video capture. For example, the video may be time-sliced to acquire individual images from the video.

The application may include an augmented reality (AR) guide or other instructions to aid the user in proper image acquisition. For example, the AR guide may include lines that illustrate where the user should position their face and eyes to acquire a proper image.

The application may then analyze the skin image. For example, the application may process the skin image into fundamental components to extract meaningful information. The image analysis may include tasks such as finding shapes, detecting edges, removing noise, counting objects, calculating statistics for texture analysis or image quality, and/or the like.

Region analysis may be used to extract statistical data and interpret that data to determine skin characteristics of the user. For example, feature extraction may be used to extract/identify features from raw data of the skin image. The application may use classification techniques to identify a set of categories (e.g., acne, wrinkles, etc.) and assign the identified features to their respective categories.

For example, image processing algorithms that may be used to identify acne may include thresholding, blob detection, Hough transform, template matching, and the like. Thresholding may include a method where pixels with intensities above or below a certain threshold are classified as spots, e.g., acne. Blob detection algorithms, such as the Laplacian of Gaussian (LoG) or Difference of Gaussians (DoG), may identify spots by determining regions with high intensity variations. Hough Transform may be adapted to detect circular spots or ellipses in an image. Such closed loops in the image, if below a threshold size, may be assessed as acne, for example. Template matching may include comparing one or more predefined acne templates with regions of the image. In examples where a match is found, the match confidence may indicate the presence of acne.

For example, image processing algorithms that may be used to identify wrinkles may include Hough transform, edge detection, Radon transform, Line Segment Detector (LSD), and the like. The Hough Transform may include techniques for detecting lines in images. The Hough transform may identify lines by converting them into points in a parameter space, where intersecting lines correspond to peaks. Edge detection algorithms, such as the Canny edge detector, can be used to find edges in an image, which can then be linked to wrinkles and facial lines. The Radon transform may be used for detecting lines, particularly in skin images with complex pigmentation. The Radon transform may be used to calculate a sum of pixel values along different angles to find wrinkles. LSD is an algorithm specifically designed for detecting line segments in images, and it may be used to identify wrinkles that vary in thicknesses as imaged. By passing the image through one or more of the algorithms, the image may be reduced to a number of identified skin elements, each skin element being characterized by, for example, category, location, size, and the like. The number and size of elements normalized by the total analyzed area may be used to assess the user's skin. The assessment may be calculated as an overall score of the user's skin health.

As illustrated, the UI may display the overall score (e.g., 7.4 out of 10) of the user's skin health and a daily insights report 102. The UI may display a user profile icon 108 that, when selected, may allow the user to customize their profile, set goals, control permissions and notifications, and/or the like. Each user may have an individual profile/account associated with only that user. Users may switch between profiles/accounts by inputting login credentials associated with the desired account. Accordingly, multiple users may have their own accounts with separate data (e.g., even if the users access the application via the same device).

The overall score may be depicted using a graphical representation 104, which may have several sections 106 that represent a plurality of skin characteristics. The skin characteristics may be a feature or quality belonging to the user's skin. For example, the plurality of skin characteristics may include clear skin, dark circles, wrinkles, fine lines, dark spots, redness, smoothness, and/or the like.

A skin characteristic may represent at least one of a region of interest, a skin element, a magnitude, and/or a timestamp. For example, the region or interest may be any of face, nose, chin, and/or cheek. The skin element may be a variable associated with the skin characteristic. For example, the skin element may be a blackhead, white head, red region, or raised bump. The magnitude may include a density metric of the skin element within the region of interest, and the timestamp may represent a time at which the skin image was received.

The application may be configured to determine scores for the skin characteristics. In some examples, the overall score may be an average of the skin characteristic scores. In some examples, the overall score may be a weighted average of the skin characteristic scores.

The application may be configured to determine scores for variables associated with the skin characteristics. The variables may be short-term variables. A variable may be linked to a particular skin characteristic. That is, the combination of the variables may be used to form a long-term skin characteristic score. For example, variables of the clear skin characteristic may include whether the user has blackheads, white heads, red bumps, raised bumps, and/or other such skin lesions. In an example, the user may have few blackheads, but many white heads and red bumps on a given day. The user may start a treatment routine to treat the white heads and red bumps. As a result, the clear skin characteristic score may increase over time as the number of white heads and red bumps is reduced. An artificial intelligence and/or machine learning (AI/ML) skin analysis may be used to count the number of skin lesions. The clear skin characteristic may include information related to the size of pores.

As another example, variable(s) of the dark spots skin characteristic may include a number of moles and/or a rating of pigmentation over time. As another example, variable(s) of the fine lines and wrinkles skin characteristics may include a number of the fine lines/wrinkles and/or a severity of the fine lines/wrinkles.

In some examples, the daily insights report 102 may include recommendations of a skin care routine and/or topical medications or products based on the current skin characteristics. The application may recommend products based on answers to a skin care questionnaire. As illustrated, the UI may include a commerce function 110 that may be used to purchase products. The products may be recommended based on the user's current skin characteristic scores. The products may be recommended based on a primary skin concern or goal of the user. In some examples, the user may use the device to capture images of products used in the routine. The UI may then provide the user with a link to order more of the products when the user runs out. In some examples, the user may enter an ingredient (e.g., rather than a product) into the application. The application may list the potential benefits and/or risks of the ingredient. The application may list products in which the ingredient can be found. In some examples, the user may provide time-dependent reports about the user's skin care routine, and the routine may be adjusted periodically based on observed changes to the skin characteristics.

The user may provide a self-assessment of current skin characteristics, and may define skin goals (e.g., desired changes in one or more skin characteristics). In some examples, the user may identify long term skin appearance concerns. AI/ML image analysis may be used to determine scores for subsets of skin characteristics and/or variables. The application may then compare the AI/ML-derived scores to the user-defined subjective scores and adjust the scores based on the comparison.

In some examples, AI/ML image analysis may be used to identify skin type(s) (e.g., sensitive, oily, etc.) of the user. The user may provide a self-assessment of their skin type. The application may identify a list of ingredients that are potential allergens or irritants. The application may identify the list of ingredients based on the user's skin type.

In some examples, the AI/ML image analysis may be used to determine skin dynamics. For example, the AI/ML image analysis may acquire a first skin image in which the user is not smiling. The AI/ML image analysis may acquire a second skin image in which the user is smiling. The AI/ML image analysis may determine, for example, how a product has reduced the appearance of wrinkles and/or fine lines when the user smiles.

The user may self-report lifestyle variables (e.g., amount of sleep, exercise, stress) that may be correlated with skin characteristics. The UI may include an indication of ambient weather conditions, which may affect the appearance of the user's skin. For example, the UI may indicate that it is very hot outside, and that the user should focus on staying hydrated. As another example, the UI may indicate that the air quality is poor, and that the user should avoid going outside.

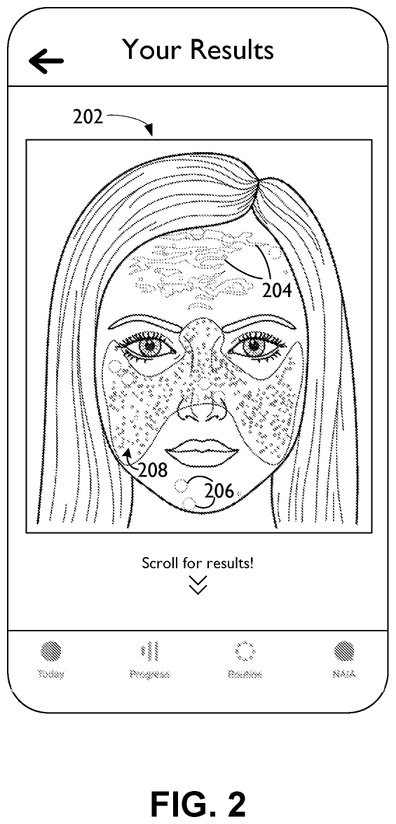

As illustrated in FIG. 2, the UI may display a skin image captured by the user with an analysis overlay 202. The analysis overlay 202 may include markings (e.g., lines, circles, and/or dots) that indicate the location of features of the user's skin that contributed to each of the skin characteristics scores. For example, the lines 204 may be a first color and may indicate wrinkles on the user's forehead. Similarly, the circles 206 may be a second color to indicate the presence of red bumps on the user's skin and the dots 208 may be a third color to indicate the presence of blackheads.

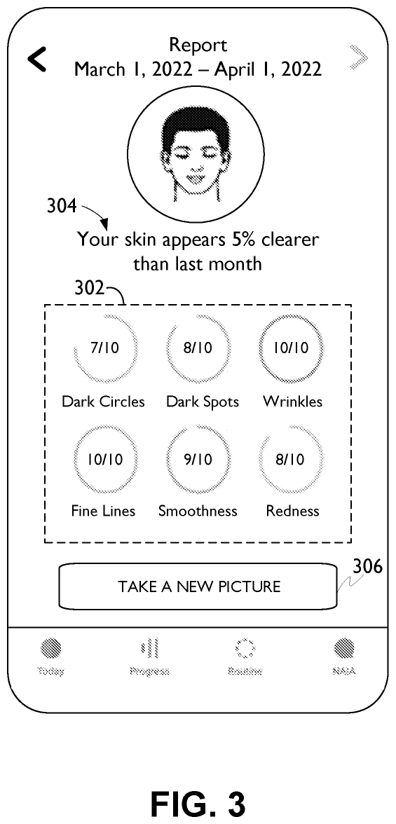

As illustrated in FIG. 3, the UI may display a report of the progress made over a period of time (e.g., over a day, week, or month). The report may include a progress bar 302 for each of the skin characteristics. In some examples, the UI may display a progress bar for a primary goal of the user (e.g., clearer skin, fewer wrinkles). The report may include an evaluation 304 of the skin characteristics that indicates improvements or diminishment of the user's skin health. For example, the evaluation 304 may state “Your skin appears 5% clearer than last month,” or “Your skin is 18% smoother than last month.” The UI may display a button 306 to allow the user to take a new skin image. The new skin image may then be analyzed, and the data from the analysis may be incorporated into the report.

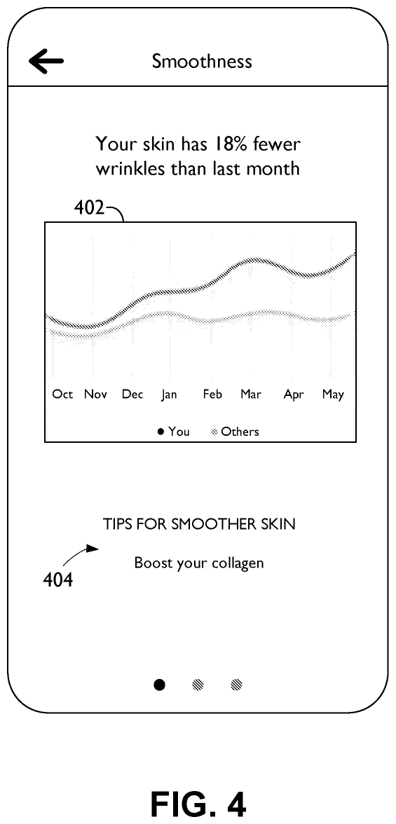

As illustrated in FIG. 4, the UI may display a weekly progress graph 402 that illustrates the scores over time. For example, the weekly progress graph 402 may be a timeline graph, a bar graph, or any other graph suitable for illustrating data over time. The progress graph 402 may illustrate the progress for a particular skin characteristic (e.g., smoothness), for a subgrouping of skin characteristics, or for a combination of skin characteristics (e.g., represented by the overall score). Reports of skin characteristic scores, graphical representations of the skin characteristics, and AR/ML image presentations of images associated with skin characteristics may be presented in time series.

The UI may display tips 404 for improving a particular skin characteristic. For example, the UI may display “for smoother skin, boost your collagen” or “to reduce the appearance of wrinkles, limit exposure to the sun.”

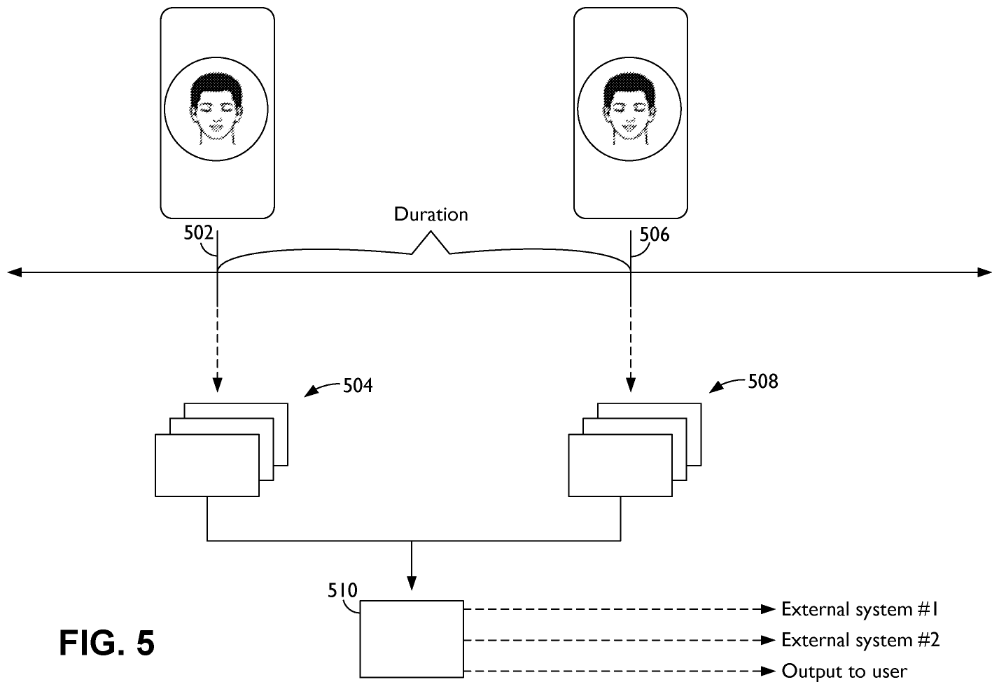

The user may request that a report containing skin characteristics information collected over time be transmitted to one or more receivers. For example, the user may request that the skin characteristics be transmitted to a skin care specialist (e.g., a dermatologist or aesthetician). For example, as illustrated in FIG. 5, the user may take a first skin image at a first time 502. The application may be configured to receive the first skin image and determine a plurality of skin characteristics 504 from the first skin image. After a duration of time, the user may take a second skin image at a time 506, and the application may receive the second skin image and determine another plurality of skin characteristics 508 from the second skin image.

The user may request that the application combine one or more skin characteristics of the plurality of skin characteristics 504 and 508 to generate an analysis output 510. The user may request that the application transmit the analysis output 510 to a skin care specialist (e.g., a dermatologist or aesthetician).

The contents of the analysis output 510 may be based on an analysis configuration. The analysis configuration may be a default analysis configuration. The analysis configuration may depend on setting(s) selected by the user. The analysis configuration may depend on setting(s) selected by the skin care specialist. For example, based on the analysis configuration, the analysis output 510 may include information associated with a particular subgrouping of skin characteristics (e.g., the report may include only information related to the clear skin and smoothness characteristics).

The analysis configuration may not affect how the analysis of the skin image is performed. The analysis configuration may dictate what the analysis output 510 looks like (e.g., the contents and/or layout of the analysis output 510). For example, the analysis configuration may indicate that a particular user prefers the analysis output to include raw data in a table format, a graphical depiction of the analysis results, an AI overlay of identified skin characteristics, skin scores, and/or the like.

In some cases, the application may be configured to receive user input that identifies the skin care specialist. The selected analysis configuration may be selected from a plurality of analysis configurations based on the user input. The user input may include an area of concern to the user (e.g., a desire to reduce acne). The application may be configured to identify an appropriate analysis configuration and/or skin care specialist based on the area of concern. In some examples, the selected analysis configuration may be based on preferences of the skin care specialist or user. In some examples, the analysis output 510 may include images that are linked to the skin characteristics that are included in the analysis output 510.

The analysis output 510 may have a different appearance and/or layout than report(s) provided to the user (e.g., via the UI). For example, report(s) provided to the user may include a list of topics about which to consult a skin care specialist (e.g., techniques for the user to better hydrate the user's skin). For example, the report(s) provided to the user may include scores for the skin characteristics and tips on improving skin characteristics. In some examples, the report(s) provided to the user may include information regarding the underlying science and/or causes of low skin scores.

For report(s) provided to a skin care specialist, the analysis output 510 may be a historical summary of data collected over the duration that includes more detailed information that the skin care specialist can use to diagnose and treat the user. In some examples, the historical summary may represent a difference between a first skin characteristic associated with the first time 502 and a second skin characteristic associated with the second time 506. In this case, the first skin characteristic and the second skin characteristic may correspond (e.g., overlap) in region of interest and skin element.

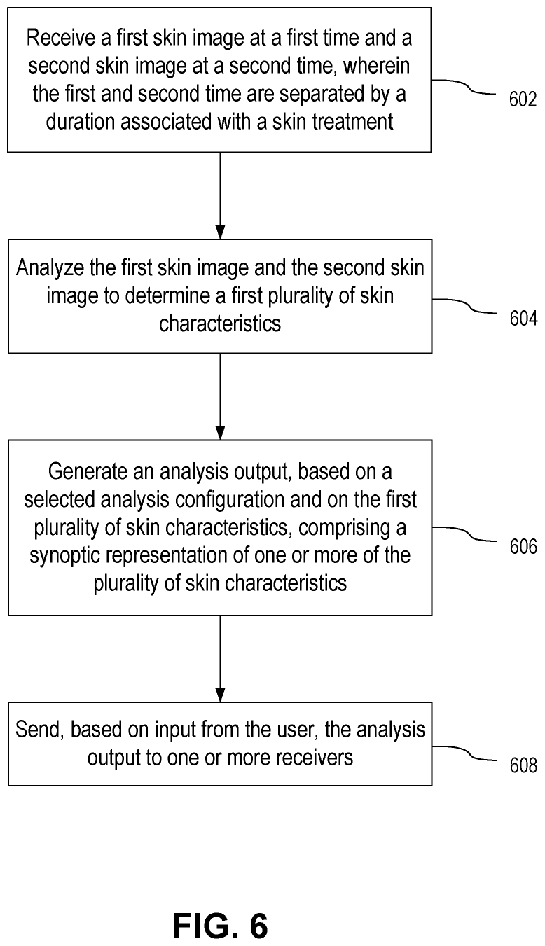

FIG. 6 is a flow diagram illustrating an example computer-implemented method for monitoring skin over time. At 602, the application may receive a first skin image at a first time (e.g., the first time 502) and a second skin image at a second time (e.g., the second time 506). The first and second time may be separated by a duration associated with a skin event (e.g., a skin treatment, exposure to sunlight, sleep, and/or any other event which may impact the appearance of the user's skin). At 604, the application may determine a plurality of skin characteristics (e.g., the skin characteristics 504 and 508) from the first skin image and the second skin image.

At 606, the application may be configured to generate an analysis output (e.g., the analysis output 510). For example, the application may be configured to generate the analysis output based on an analysis configuration and the plurality of skin characteristics. For example, as explained herein, the analysis configuration may determine which of the skin characteristics to include in the analysis output. The analysis output may include a synoptic representation of one or more of the plurality of skin characteristics. The analysis configuration may determine how the synoptic representation is presented to the user and/or skin care specialist.

At 608, the application may be configured to transmit the analysis output to one or more receivers. For example, the application may be configured to transmit the analysis output to a skin care specialist or other receiver(s) (e.g., a health care provider, such as a primary care physician). The skin care specialist may be selected based on user input(s) or based on the selected analysis configuration. As another example, the application may be configured to transmit the analysis output to memory. For example, the application may be configured to save the analysis output as an image file. In some examples, the application may be configured to upload the image file to a patient portal associated with the skin care specialist. In some examples, the user may transmit the analysis output directly to the one or more receivers. In some examples, the analysis output may be transmitted to the one or more receivers through a telemedicine operator (e.g., an operator associated with the skin care specialist).

An example method (e.g., such as that described with respect to FIG. 6) may be computer-implemented by any suitable structure, such as structure suitable for reception, processing, and outputting of data, for example. The example method may be performed by a device or tool, such as a mobile device, smartphone, tablet, computer, laptop, application, processor, or any other suitable piece of equipment, hardware, firmware, and/or software capable of performing the techniques described herein, for example.

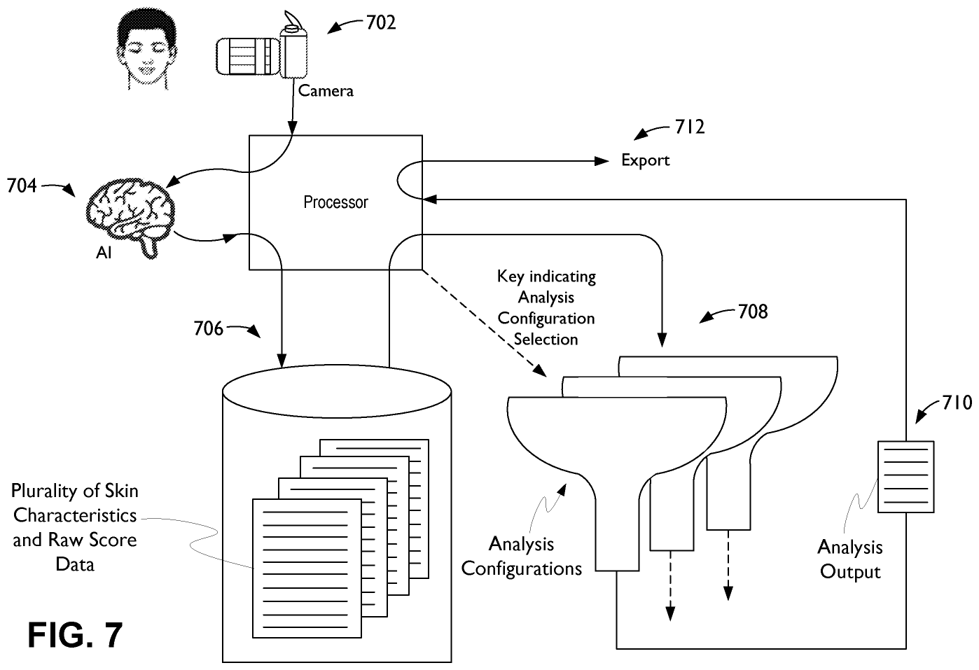

FIG. 7 is a block diagram illustrating an example generation of an analysis output based on longitudinal information about one or more skin characteristics. At 702, the camera may be used to capture one or more skin image(s). The skin image(s) may be associated with a user of the application. The skin image(s) may then be sent to a processor (e.g., associated with the application).

At 704, the processor may send the skin images to an AI system. The AI system may be a standalone system or may be a part of the application. The AI system may be a machine learning system (e.g., an AI/ML system). Machine learning is a branch of AI that seeks to build computer systems that may learn from data without human intervention. These techniques may rely on the creation of analytical models that may be trained to recognize patterns within a dataset, such as a data collection. These models may be deployed to apply these patterns to data, such as biomarkers, to improve performance without further guidance.

The AI system may compare the skin image(s) to datapoints from a database on skin images. For example, the database of skin images may include image-score pairs, in which each image has been scored by a dermatologist. In some examples, the datapoints may be associated with skin characteristics of the skin images. Based on the comparison, the AI system may generate scores for a plurality of skin characteristics associated with the skin image(s).

The scores may be generated based on the comparison of the captured skin image to the datapoints from the database. For example, the skin image may be compared to datapoints representing high and low scores for certain skin characteristics, and the scores for the skin image may be generated based on the datapoints to which the skin image most closely corresponds.

The AI/ML system may be trained. Machine learning may be supervised (e.g., supervised learning) or unsupervised (e.g., unsupervised learning). The AI system may be trained, for example, to give a lower score if a large number of an undesirable skin characteristics (e.g., acne or wrinkles) are present. The AI system may be further trained based on previous scores given to the user. For example, the AI system may determine that a current score should go up relative to a score for a previous skin image if there are relatively fewer undesirable skin characteristics than the previous skin image and/or if the severity of those undesirable skin characteristics has lessened.

A supervised learning algorithm may create a mathematical model from training a dataset (e.g., training data). The training data may consist of a set of training examples. A training example may include one or more inputs and one or more labeled outputs. The labeled output(s) may serve as supervisory feedback. In a mathematical model, a training example may be represented by an array or vector, sometimes called a feature vector. The training data may be represented by row(s) of feature vectors, constituting a matrix. Through iterative optimization of an objective function (e.g., cost function), a supervised learning algorithm may learn a function (e.g., a prediction function) that may be used to predict the output associated with one or more new inputs. A suitably trained prediction function may determine the output for one or more inputs that may not have been a part of the training data. Example algorithms may include linear regression, logistic regression, and neutral network. Example problems solvable by supervised learning algorithms may include classification, regression problems, and/or the like.

An unsupervised learning algorithm may train on a dataset that includes inputs. The unsupervised learning algorithm may find a structure in the data. The structure in the data may be similar to a grouping or clustering of data points. As such, the algorithm may learn from training data that may not have been labeled. Instead of responding to supervisory feedback, an unsupervised learning algorithm may identify commonalities in training data and may react based on the presence or absence of such commonalities in each train example. Example algorithms may include Apriori algorithm, K-Means, K-Nearest Neighbors (KNN), K-Medians, and/or the like. Example problems solvable by unsupervised learning algorithms may include clustering problems, anomaly/outlier detection problems, and/or the like.

Machine learning may include reinforcement learning. Reinforcement learning may be an area of machine learning concerned with how software agents take actions in an environment to maximize a notion of cumulative reward. Reinforcement learning algorithms may not assume knowledge of an exact mathematical model of the environment (e.g., represented by a Markov decision process (MDP)) and may be used when exact models may not be feasible. Reinforcement learning algorithms may be used in autonomous vehicles or in learning to play a game against a human opponent, for example.

Machine learning may be a part of a technology platform called cognitive computing (CC), which may constitute various disciplines such as computer science and cognitive science. CC systems may be capable of learning at scale, reasoning with purpose, and interacting with humans naturally. By means of self-teaching algorithms that may use data mining, visual recognition, and/or natural language processing, a CC system may be capable of solving problems and optimizing human processes.

The output of machine learning's training process may be a model for predicting outcome(s) on a new dataset. For example, a linear regression learning algorithm may be a cost function that may minimize the prediction errors of a linear prediction function during the training process by adjusting the coefficients and constants of the linear prediction function. If a minimum error is reached, the linear prediction function with adjusted coefficients may be deemed trained and constitute the model the training process has produced. For example, a neural network (NN) algorithm (e.g., multilayer perceptrons (MLP)) for classification may include a hypothesis function represented by a network of layers of nodes that are assigned with biases and interconnected with weight connections. The hypothesis function may be a non-linear function (e.g., a highly non-linear function) that may include linear functions and logistic functions nested together with the outermost layer consisting of one or more logistic functions. The NN algorithm may include a cost function to minimize classification errors (e.g., by adjusting the biases and weights through a process of feedforward propagation and backward propagation). If a global minimum is reached, the optimized hypothesis function with its layers of adjusted biases and weights may be deemed trained and constitute the model the training process has produced.

Data collection may be performed for machine learning as a first stage of the machine learning lifecycle. Data collection may include steps such as identifying various data sources, collecting data from the data sources, integrating the data, and/or the like. For example, for training a machine learning model for predicting surgical complications and/or post-surgical recovery rates, data sources containing pre-surgical data, such as a patient's medical conditions and biomarker measurement data, may be identified. Such data sources may be a patient's electronic medical records (EMR), a computing system storing the patient's pre-surgical biomarker measurement data, and/or other like datastores. The data from the data sources may be retrieved and stored in a central location for further processing in the machine learning lifecycle. The data from the data sources may be linked (e.g., logically linked). The data may be accessed as if it were centrally stored. Surgical data and/or post-surgical data may be similarly identified and/or collected. The collected data may be integrated (e.g., combined). For example, a patient's pre-surgical medical record data, pre-surgical biomarker measurement data, pre-surgical data, surgical data, and/or post-surgical may be combined into a record for the patient. The record for the patient may be an EMR.

Data preparation may be performed for machine learning as another stage of the machine learning lifecycle. Data preparation may include data preprocessing steps such as data formatting, data cleaning, and data sampling. For example, the collected data may not be in a data format suitable for training a model. In an example, a patient's integrated data record of pre-surgical EMR record data and biomarker measurement data, surgical data, and post-surgical data may be in a rational database. Such data record may be converted to a flat file format for model training. In an example, the patient's pre-surgical EMR data may include medical data in text format, such as the patient's diagnoses of emphysema, pre-operative treatment (e.g., chemotherapy, radiation, blood thinner), and/or the like. The data may be mapped to numeric values for model training. For example, the patient's integrated data record may include personal identifier information or other information that may identify a patient (e.g., age, an employer, a body mass index (BMI), demographic information, and/or the like). Such identifying data may be removed before model training. For example, identifying data may be removed for privacy reasons. As another example, data may be removed because there is more data available than can be used for model training. In this case, a subset of the available data may be randomly sampled and selected for model training and the remainder may be discarded.

Data preparation may include data transforming procedures (e.g., after preprocessing), such as scaling and aggregation. For example, the preprocessed data may include data values in a mixture of scales. These values may be scaled up or down, for example, to be between 0 and 1 for model training. For example, the preprocessed data may include data values that carry more meaning when aggregated. In an example, there may be multiple prior colorectal procedures a patient has had. The total count of prior colorectal procedures may be more meaningful for training a model to predict surgical complications due to adhesions. In such case, the records of prior colorectal procedures may be aggregated into a total count for model training purposes.

Model training may be another aspect of the machine learning lifecycle. The model training process as described herein may be dependent on the machine learning algorithm used. A model may be deemed suitably trained after it has been trained, cross validated, and tested. Accordingly, the dataset from the data preparation stage (e.g., an input dataset) may be divided into a training dataset (e.g., 60% of the input dataset), a validation dataset (e.g., 20% of the input dataset), and a test dataset (e.g., 20% of the input dataset). After the model has been trained on the training dataset, the model may be run against the validation dataset to reduce overfitting. If accuracy of the model were to decrease when run against the validation dataset when accuracy of the model has been increasing, this may indicate a problem of overfitting. The test dataset may be used to test the accuracy of the final model to determine whether it is ready for deployment or if more training is required.

Model deployment may be another aspect of the machine learning lifecycle. The model may be deployed as a part of a standalone computer program. The model may be deployed as a part of a larger computing system. A model may be deployed with model performance parameter(s). The performance parameters may monitor the model accuracy as the model is making predictions based on a dataset in production. For example, the performance parameters may keep track of false positives and false negatives for a classification model. The performance parameters may store the false positives and false negatives for further processing to improve the model's accuracy.

Post-deployment model updates may be another aspect of the machine learning cycle. For example, a deployed model may be updated as false positives and/or false negatives are predicted on production data. In an example, for a deployed MLP model for classification, as false positives occur, the deployed MLP model may be updated to increase the probability cutoff for predicting a positive to reduce false positives. In an example, for a deployed MLP model for classification, as false negatives occur, the deployed MLP model may be updated to decrease the probability cutoff for predicting a positive to reduce false negatives. In an example, for a deployed MLP model for classification of surgical complications, as false positives and false negatives occur, the deployed MLP model may be updated to decrease the probability cutoff for predicting a positive to reduce false negatives (e.g., because it may be less critical to predict a false positive than a false negative).

A deployed model may be updated as more live production data becomes available as training data. In such cases, the deployed model may be further trained, validated, and tested with the additional live production data. In an example, the updated biases and weights of a further-trained MLP model may update the deployed MLP model's biases and weights. Those skilled in the art will recognize that post-deployment model updates may not be a one-time occurrence and may occur as frequently as suitable for improving the deployed model's accuracy.

At 706, the plurality of skin characteristics and associated scores (e.g., raw score data) may be stored in any suitable form of memory. As illustrated, the raw data may be extensive and, therefore, may include more information than is useful to the user and/or skin care specialists.

In some examples, the processor may retrieve the raw data, which may be filtered using one or more analysis configurations at 708. For example, a first analysis configuration may be associated with a first skin care specialist and a second analysis configuration may be associated with a second, different skin care specialist.

As an example, the first skin care specialist may specialize in acne treatment, while the second skin care specialist may specialize in treatment for wrinkles and fine lines. Accordingly, the first analysis configuration may filter the raw data so that the information sent to the first skin care specialist only includes information that is relevant to treating acne, while the second analysis configuration may filter the raw data so that the information sent to the second skin care specialist only includes information that is relevant to treating wrinkles and fine lines.

The analysis configurations may determine how the analysis output is formatted. For example, some skin care specialists may prefer that skin images (and/or skin images with overlays such as the overlay 302 in FIG. 3) be included in the analysis output, while other skin care specialists may prefer that only data be included in the analysis output and/or that the data be organized in a particular way.

The processor may use a key to indicate which of the analysis configurations to use to generate the analysis output. For example, the processor may receive user input(s) indicating that the user is concerned about reducing the severity of wrinkles on the user's forehead. The processor may use the user input to generate a key that corresponds to (or indicates) an analysis configuration that will generate an analysis output with information that may help a skin care specialist counsel the patient on the most effective ways to reduce the appearance of wrinkles.

One or more of the analysis configurations may be used to generate one or more output(s) for the user that captured the skin image(s). As explained herein, in some cases, the analysis output(s) generated for the skin care specialist(s) may have a different appearance and/or layout than output(s) provided to the user.

As illustrated, at 710, an analysis output may be generated and sent to the processor. The processor may export the analysis output at 712. In some examples, exporting the analysis output may involve converting the analysis output to another format (e.g., an image file format, portable document format (PDF), and/or the like). In some examples, exporting the analysis output may involve uploading the analysis output (or the converted version of the analysis output) to a health care portal associated with a skin care specialist. The processor may be configured to convert the analysis output to a file format that is compatible with the file requirements of a selected health care portal.

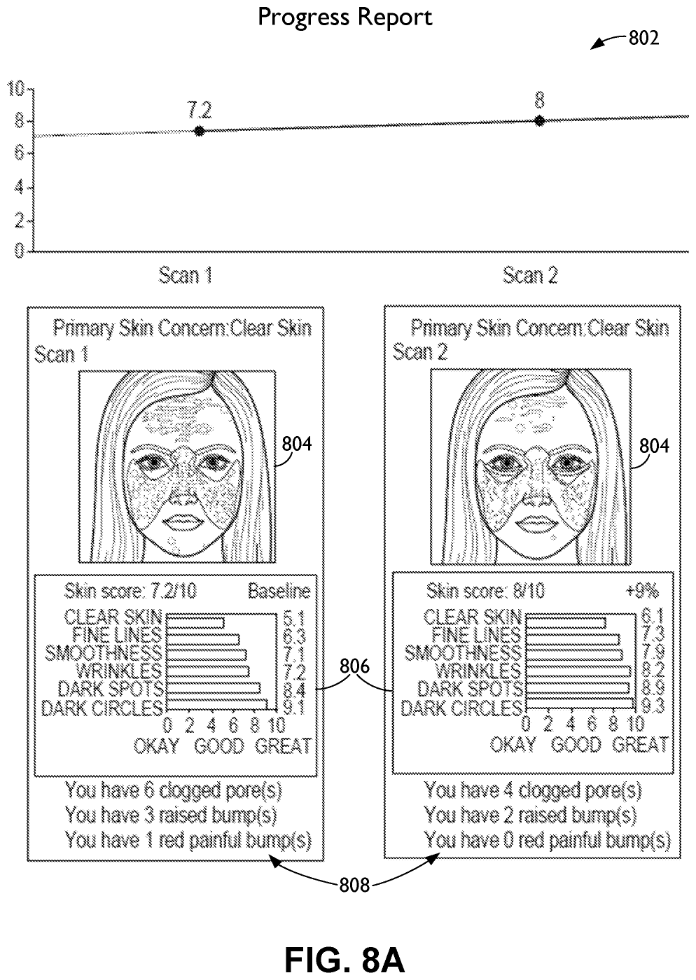

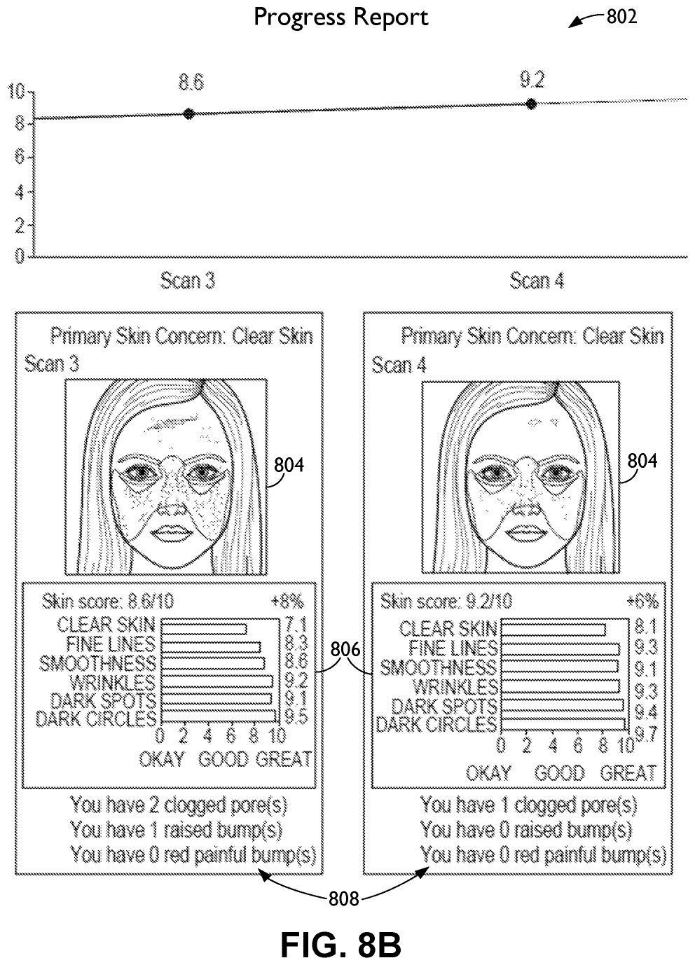

FIGS. 8A and 8B illustrate an example analysis output. The analysis output may include a timeline 802 illustrating the user's progress. For example, as shown, the timeline 802 may show the overall scores associated with skin images taken by the user over time. The analysis output may include the skin images 804 associated with each of the overall scores. The analysis output may allow a skin care specialist to easily perform follow-ups with a patient. For example, the timeline 802 and skin images 804 may allow a skin care specialist to analyze the patient's progress (e.g., easily and quickly) over time (e.g., since a previous report, a treatment, and/or appointment with the skin care specialist).

The analysis output may include scores 806 for each of a plurality of skin characteristics. The skin characteristic scores 806 may allow a skin care specialist to better analyze the user's skin concern(s). For example, as illustrated, the user's primary skin concern may be clear skin. Accordingly, the skin care specialist may focus on the clear skin score to better analyze how particular treatment(s) have affected the user's clear skin score over time.

The analysis output may include notes 808 about the skin variables associated with the primary skin concern and/or other skin characteristics. For example, the analysis output may include a list of information regarding the skin variables, such as the number of clogged pores, raised bumps, red painful bumps, etc. that were detected in a given skin image 804.

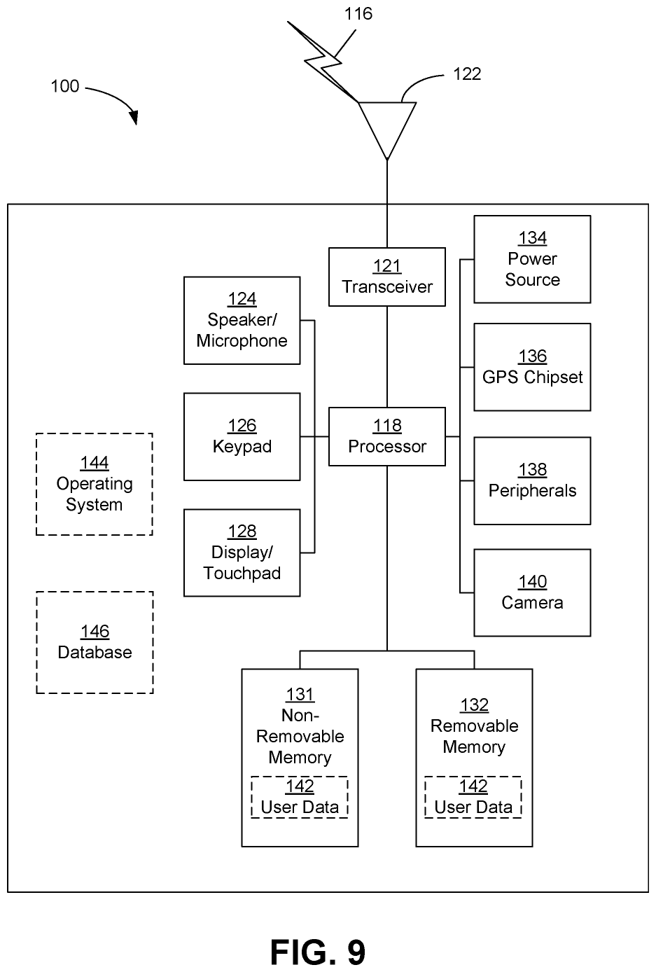

FIG. 9 is a system diagram illustrating an example computing device 100, which may be used to monitor skin over time. The computing device 100 may be, for example, a cellular phone, tablet, or other such device. As shown in FIG. 9, the computing device 100 may include a processor 118, a transceiver 121, a transmit/receive element 122, a speaker/microphone 124, a keypad 126, a display/touchpad 128, non-removable memory 131, removable memory 132, a power source 134, a global positioning system (GPS) chipset 136, peripherals 138, a camera 140, an operating system 144, and/or a database 146, among others. It will be appreciated that the computing device 100 may include any sub-combination of the foregoing elements while remaining consistent with an embodiment.

The processor 118 may be a general-purpose processor, a special-purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGA) circuits, any other type of integrated circuit (IC), a state machine, and/or the like. The processor 118 may perform signal coding, data processing, power control, input/output processing, and/or any other functionality that enables the computing device 100 to operate in a wireless environment. The processor 118 may be coupled to the transceiver 121, which may be coupled to the transmit/receive element 122. While FIG. 9 depicts the processor 118 and the transceiver 121 as separate components, a person of ordinary skill in the art will appreciate that the processor 118 and the transceiver 121 may be integrated together in an electronic package or chip.

In some examples, the transceiver 121 and the transmit/receive element 122 may be used to transmit analysis output(s) to one or more skin care specialists. The transmit/receive element 122 may be configured to transmit signals to, or receive signals from, a base station over an air interface 116. For example, the transmit/receive element 122 may be an antenna configured to transmit and/or receive radio frequency (RF) signals. The transmit/receive element 122 may be an emitter/detector configured to transmit and/or receive infrared (IR), ultraviolet (UV), or visible light signals, for example. The transmit/receive element 122 may be configured to transmit and/or receive both RF and light signals. A person of ordinary skill in the art will appreciate that the transmit/receive element 122 may be configured to transmit and/or receive any combination of wireless signals.

The processor 118 of the computing device 100 may be coupled to, and may receive user input data from, the speaker/microphone 124, the keypad 126, and/or the display/touchpad 128 (e.g., a liquid crystal display (LCD) display unit or organic light emitting diode (OLED) display unit). The processor 118 may output user data to the speaker/microphone 124, the keypad 126, and/or the display/touchpad 128. The processor 118 may access information from, and store data in, any type of suitable memory, such as the non-removable memory 131 and/or the removable memory 132. The non-removable memory 131 may include random-access memory (RAM), read-only memory (ROM), a hard disk, or any other type of memory storage device. The removable memory 132 may include a subscriber identity module (SIM) card, a memory stick, a secure digital (SD) memory card, and/or the like. As illustrated, user data 142 may be stored in the non-removable memory 131 and/or the removable memory 132. The user data may include the raw data associated with the skin characteristics, data regarding preference(s) of the user (e.g., for use in determining an analysis configuration), and/or the like. The processor 118 may access information from, and store data in, memory that is not physically located on the computing device 100, such as on a server or a home computer (not shown).

The operating system 144 may be single-tasking or multi-tasking, and may manage the functions of the processor 118. For example, the operating system 144 may handle inputs and outputs to and from the processor 118, schedule tasks to be performed by the processor 118, and/or perform other such management functions. The operating system 144 may manage the non-removable memory 131 and/or the removable memory 132. For example, the operating system 144 may determine which type of memory will be used to store different datasets.

The processor 118 may receive power from the power source 134 and may be configured to distribute and/or control the power to the other components in the computing device 100. The power source 134 may be any suitable device for powering the computing device 100. For example, the power source 134 may include one or more dry cell batteries (e.g., nickel-cadmium (NiCd), nickel-zinc (NiZn), nickel metal hydride (NiMH), lithium-ion (Li-ion), etc.), solar cells, fuel cells, and/or the like.

The processor 118 may be coupled to the GPS chipset 136, which may be configured to provide location information (e.g., longitude and latitude) regarding the current location of the computing device 100. In addition to, or in lieu of, the information from the GPS chipset 136, the computing device 100 may receive location information over the air interface 116 from a base station and/or determine its location based on the timing of the signals being received from two or more nearby base stations. A person of ordinary skill in the art will appreciate that the computing device 100 may acquire location information by way of any suitable location-determination method while remaining consistent with an embodiment.

The processor 118 may be coupled to other peripherals 138, which may include one or more software and/or hardware modules that provide additional features, functionality and/or wired or wireless connectivity. For example, the peripherals 138 may include an accelerometer, an e-compass, a satellite transceiver, a universal serial bus (USB) port, a vibration device, a television transceiver, a hands-free headset, a Bluetooth® module, a frequency modulated (FM) radio unit, a digital music player, a media player, a video game player module, an Internet browser, a Virtual Reality and/or Augmented Reality (VR/AR) device, an activity tracker, and/or the like.

The peripherals 138 may include one or more sensors, the sensors may be one or more of a gyroscope, an accelerometer, a hall effect sensor, a magnetometer, an orientation sensor, a proximity sensor, a temperature sensor, a time sensor, a geolocation sensor, an altimeter, a light sensor, a touch sensor, a barometer, a gesture sensor, a biometric sensor, a humidity sensor, and/or the like.

The processor 118 may be coupled to the camera 140. In some examples, the camera 140 may be used to capture skin images of a user. The camera 140 may transmit the image to the processor 118 (or an AI system such as that discussed with respect to FIG. 7) to determine a plurality of skin characteristics from the first skin image and the second skin image. The skin characteristics and associated scores may be determined by comparing the captured skin images to skin images in a database, such as the database 146. The processor 118 may be configured to generate a report (e.g., an analysis output) based on one or more of the skin characteristics, and display (e.g., via the display/touchpad 128) the report on a mobile application UI, such as those illustrated in FIGS. 1-4.

1. A device, comprising:
a processor configured to:
receive a first skin image at a first time and a second skin image at a second time, wherein the first skin image and the second skin image are associated with a user, and wherein the first and second time are separated by a duration associated with a skin event;
determine a plurality of skin characteristics from the first skin image and the second skin image;
generate an analysis output, based on an analysis configuration and on the plurality of skin characteristics, comprising a synoptic representation of one or more of the plurality of skin characteristics associated with an area of concern of the user; and
transmit the analysis output to one or more receivers comprising a skin care specialist, wherein the synoptic representation presents a historical summary of the one or more of the plurality of skin characteristics in a form suitable to the skin care specialist.

2. The device of claim 1, wherein the processor is further configured to receive user input that identifies the skin care specialist, and wherein the analysis configuration is selected from a plurality of analysis configurations based on the user input.

3. The device of claim 1, wherein a skin characteristic of the plurality of skin characteristics represents at least a skin element and a score associated with the skin element.

4. The device of claim 1, wherein the analysis output comprises a historical summary, over the duration, of the plurality of skin characteristics, wherein the historical summary represents a difference between a first skin characteristic associated with first time and a second skin characteristic associated with second time.

5. The device of claim 4, wherein the first skin characteristic and the second skin characteristic correspond in region of interest and skin element.

6. The device of claim 1, wherein the synoptic representation is a first synoptic representation, and wherein the processor is further configured to:
generate a second analysis output, based on a second analysis configuration and on the plurality of skin characteristics, comprising a second synoptic representation, different from the first synoptic representation, of one or more of the plurality of skin characteristics; and
transmit, based on input from the user, the second analysis output to one or more other receivers.

7. The device of claim 6, wherein the first synoptic representation comprises a synoptic representation of a first subset of the plurality of skin characteristics, and wherein the second synoptic representation comprises a synoptic representation of a second subset of the plurality of skin characteristics different from the first subset.

8. The device of claim 1, wherein the processor is further configured to:
save the analysis output as an image file; and
upload the image file to a patient portal associated with the skin care specialist.

9. The device of claim 1, wherein the synoptic representation comprises at least one of the first skin image and the second skin image with an overlay of markings illustrating one or more of the plurality of skin characteristics.

10. The device of claim 1, wherein the duration associated with the skin event is greater than or equal to a minimum amount of time between images, and wherein the processor is further configured to block the user from capturing the second skin image if the minimum amount of time between images has not passed since receipt of the first skin image.

11. The device of claim 1, wherein the analysis output comprises information regarding a subset of the plurality of skin characteristics, the subset being associated with one or more skin characteristics indicated by the user.

12. A method, comprising:
receiving a first skin image at a first time and a second skin image at a second time, wherein the first and second time are separated by a duration associated with a skin event;
determining a plurality of skin characteristics from the first skin image and the second skin image;
generating an analysis output, based on an analysis configuration and on the plurality of skin characteristics, comprising a synoptic representation of one or more of the plurality of skin characteristics;
determining, based on the analysis configuration, that one or more receivers of the analysis output comprise a skin care specialist; and
transmitting the analysis output to the one or more receivers, wherein the analysis output comprises a historical summary, over the duration, of the one or more of the plurality of skin characteristics, based on a preference of the skin care specialist.

13. The method of claim 12, wherein a skin characteristic of the plurality of skin characteristics represents at least a skin element and a magnitude of the skin element.

14. The method of claim 12, wherein the analysis output comprises a historical summary, over the duration, of the plurality of skin characteristics, wherein the historical summary represents a difference between a first skin characteristic associated with first time and a second skin characteristic associated with second time.

15. A non-transitory computer-readable medium comprising computer-executable instructions that when executed on a smart phone cause the smart phone to:
receive a first skin image at a first time and a second skin image at a second time, wherein the first and second time are separated by a duration associated with a skin event;
determine a plurality of skin characteristics from the first skin image and the second skin image; and
generate an analysis output, based on an analysis configuration and on the plurality of skin characteristics, comprising a synoptic representation of the plurality of skin characteristics, wherein the synoptic representation is configured to be received by a telemedicine server for presentation to a skin care specialist.

16. The computer-readable medium of claim 15, wherein the computer-executable instructions that when executed on a smart phone further cause the smart phone to receive a user input, wherein the user input comprises a skin characteristic of concern, and wherein the analysis output is based on the skin characteristic of concern.

17. The computer-readable medium of claim 15, wherein the computer-executable instructions that when executed on a smart phone further cause the smart phone to receive a user input, wherein the user input comprises a skin characteristic of concern, and wherein the skin care specialist is identified based on an expertise regarding the skin characteristic of concern.

18. The computer-readable medium of claim 15, wherein the computer-executable instructions that when executed on a smart phone further cause the smart phone to:
save the analysis output as an image file; and
upload the image file to a patient portal of the telemedicine server.

19. The computer-readable medium of claim 15, wherein a skin characteristic of the plurality of skin characteristics represents at least a skin element and a magnitude of the skin element.

20. The computer-readable medium of claim 15, wherein the analysis output comprises a historical summary, over the duration, of the plurality of skin characteristics, wherein the historical summary represents a difference between a first skin characteristic associated with first time and a second skin characteristic associated with second time.
